(19) United States
(12) Patent Application Publication
(10) Pub. No.: US 2024/0041963 A1
(43) Pub. Date: Feb. 8, 2024

(54) A NUTRACEUTICALS FORMULATION WITH AN ENHANCED ORGANOLEPTIC PROPERTIES USED FOR SCALP AND HAIR CARE

(71) Applicant: DiaBliss Consumer Products Pvt Ltd, Chennai (IN)

(72) Inventors: Vr. RAMANATHAN, Chennai (IN); Siva VALLABHANENI, Chennai (IN); V. RAJALAKSHMI, Chennai (IN)

(73) Assignee: DiaBliss Consumer Products Pvt Ltd, Chennai, TN (IN)

(21) Appl. No.: 18/258,642

(22) PCT Filed: Dec. 22, 2021
(86) PCT No.: PCT/IN2021/051194

§ 371 Date: Jun. 21, 2023

(30) Foreign Application Priority Data
Dec. 22, 2020 (IN) 202041055871

(51) Int. Cl. A61K 36/39; A61K 36/428; A61K 36/28; A61K 36/75; A61K 36/736; A61K 36/52; A61K 36/48; A61P 17/14

(52) U.S. Cl. CPC A61K 36/39; A61K 36/428; A61K 36/28; A61K 36/75; A61K 36/736; A61K 36/52; A61K 36/48; A61P 17/14

(57) ABSTRACT

Nutraceutical formulations, which could be used for scalp and hair care purposes, with enhanced organoleptie properties have elements of biological origin more particularly a herbal formulation(s).

### FIELD OF THE INVENTION

The subject matter described herein, in general, relates to a nutraceuticals formulation, which could be used for scalp and hair care purposes, with an enhanced organoleptic properties. Said formulation have elements of biological origin more particularly a herbal formulation(s).

### BACKGROUND OF THE INVENTION

Hair loss, also known as baldness or alopecia most often can be noticed on the scalp but might also be present on the entire body. This may be caused by heredity, changes in hormones, various medications and medical conditions. Everyone is at risk of hair loss, but it is most common in men. Hair loss might appear in a variety of ways, depending on the cause. There are two types of hair loss—temporary and permanent. Generally, the main symptoms are continual thinning on top of the head, patchy or circular bald spots and sudden loosening of hair. Other symptoms such as scarring, inflammation is not common. Risk factors that increase the risk of hair loss include family history, weight loss, age, stress and certain medical conditions.

Life vicissitudes, including illness, emotional trauma, protein deprivation (during strict dieting), and hormonal changes like those in pregnancy, puberty, and menopause may cause hair loss.

Several health conditions, including thyroid disease, iron deficiency anemia, and secondary syphilis, can cause hair loss

### PREVALENCE

Hair loss is a common problem worldwide, with pattern hair loss affecting around 50% of all males worldwide and a quarter of females. Hair loss may induce stress and lower self-esteem.

Most common condition was chronic telogen effluvium (CTE) (62%), followed by female pattern hair loss (FPHL) (22%) and acute telogen effluvium (ATE) (16%). Incidence of hair loss was highest in 21-40 years of age group.

Psychological and social stress on women is “immense” compared to men, which can contribute to hair loss among young women. According to the American Hair Loss Association, by the age of 35, two-thirds of American men will have some degree of appreciable hair loss, and by the age of 50, approximately 85% of men will have significant hair thinning. Some studies have estimated the prevalence of female pattern hair loss to be 6% in women less than 50 years of age and 38% in those more than 70; however, other studies have shown a prevalence as high as 32% in women more than 20 years of age.

According to trichologists female hair loss is estimated to affect around 20 per cent of Indian women before they are of the age of 30. This problem starts to increase significantly as women age and start experiencing menopause. Prevalence of hair fall among Indian Men was found to be 60.3%, prevalence of dandruff was found to be 17.1% and the prevalence of baldness was found to be 50.4%. Prevalence of greying among men aged 18-35 years was found to be 37.97%.

Currently there is no highly effective and renowned cure for hair loss, thus making the search for universal treatment a high priority.

Hair loss generally happens differently for males and females. In male-pattern hair loss (MPHL), the hair loss often presents itself as either a receding hairline, loss of hair on the crown (vertex) of the scalp or a combination of both, while in female-pattern hair loss (FPHL), it typically presents as a thinning of the hair.

### MEASUREMENT OF HAIR LOSS

### Ludwig Scale—Female Hair Loss Measurement Scale

The Ludwig hair loss scale illustrates progressive hair loss patterns in women. It defines several common types of hair loss patterns associated with female pattern baldness and can help determine level of hair loss. It is useful for purposes of diagnosis and discussion and can help determine next steps. In sum, Type 1 is mild, Type 2 is moderate and Type 3 is extensive. FIG. 1 pictorially summarizes the Ludwig scale.

### Norwood Scale—Male Hair Loss Measurement Scale

The Norwood scale (or Hamilton-Norwood scale) is the leading classification system used to measure the extent of male pattern baldness. Men typically lose their hair in one of several common patterns over the course of many decades. The Norwood scale provides easy-to-reference images that indicate different stages of balding.

The Norwood scale has seven stages. Each stage measures the severity and pattern of hair loss. FIG. 2 illustrates each of the seven stages of hair loss in men.
Stage 1. No significant hair loss or recession of the hairline.
Stage 2. There is a slight recession of the hairline around the temples. This is also known as an adult or mature hairline.
Stage 3. The first signs of clinically significant balding appear. The hairline becomes deeply recessed at both temples, resembling an M, U, or V shape. The recessed spots are completely bare or sparsely covered in hair.
Stage 3 vertex. The hairline stays at stage 2, but there is significant hair loss on the top of the scalp (the vertex).
Stage 4. The hairline recession is more severe than in stage 2, and there is sparse hair or no hair on the vertex. The two areas of hair loss are separated by a band of hair that connects to the hair remaining on the sides of the scalp.
Stage 5. The two areas of hair loss are larger than in stage 4. They are still separated, but the band of hair between them is narrower and sparser.
Stage 6. The balding areas at the temples join with the balding area at the vertex. The band of hair across the top of the head is gone or sparse.
Stage 7. The most severe stage of hair loss, only a band of hair going around the sides of the head remains. This hair is usually not dense and may be fine.

### Current Treatments for Hair Loss for Women

For woman experiencing early signs of hair loss, medications such as Minoxidil for women can have a significant long-term impact on helping keep the hair one has. Platelet rich plasma treatment has also been shown to be effective for treating hair loss in women. Other options include follicular unit extraction (FUE) where hair follicles are removed one-by-one from the donor area or by a strip harvest from the donor area where the hair is more robust and plentiful, then “planted” in the thinning areas.

If hair loss does not seem related to the Ludwig hair loss scale patterns, individuals may be suffering from traction alopecia, inflammatory conditions, or hair loss due to scarring due to injury or cosmetic procedures and interventions tend to be limited.

### Current Treatments for Hair Loss for Men

Treatments for male hair loss include the following: 1. Over the counter solutions, 2. Prescriptions, 3. Procedures
1. Over the counter solutions include:
   a. Minoxidil: It is applied directly to the scalp, this medication and can stop hairs from getting thinner It can also stimulate hair growth on the top of the scalp.
   b. Laser devices: There are various brushes, combs, and other devices that release laser light and are marketed as hair loss treatments. These devices might stimulate hair growth, . but they haven't been clinically proven to do so.
2. Prescriptions: They include, Finasteride which is a pill that's approved by the U.S. Food and Drug Administration to treat men with hair loss. According to the American Academy of Dermatology, it slows hair loss in about 88 percent of men and stimulates regrowth in about 66 percent of men. Side effects of Finasteride include, impotence, swelling in hands or feet, swelling or tenderness in breasts, dizziness, weakness, feeling like you might pass out, headache, runny nose, or skin rash.
3. Procedures: There are also medical procedures available for the treatment of hair loss, including:
   a. Hair transplantation. Parts of your scalp that have good hair growth are removed, and hair follicles are transplanted to the balding areas,
   b. Scalp reduction. Some of the bald scalp is surgically removed, and the parts of the scalp with good hair growth are brought closer together. This can be combined with a hair transplant.
   c. Scalp expansion. Devices are inserted under the scalp for about three to four weeks to stretch the skin. This procedure may be done before a scalp reduction or as a stand-alone treatment.

All the treatments available for treating hair or scalp related issues or concerns are unable to address them all in a cohesive manner. Also, the success rate has always been a grey area for all the treatments available so far. Further, the cost associated with the entire course of treatment works as a deterrent for population at large. Thus there exists a grave need for a cost effective and a holistic treatment approach for treating hair and/or scalp related illness and general issues.

### SUMMARY OF THE INVENTION

In the last three decades, a lot of concerted efforts have been channeled into researching into local plants with hair and/or scalp related illnesses. The hair and/or scalp related illnesses of some of these medicinal plants have been validated and others disproved.

It is an object of the present invention to provide for a water based herbal formulation that hair and/or scalp related illnesses among human subjects. The product formulation is prepared using the embodiments described in the Indian Patent application no: 202041041780 and the same is incorporated herewith by reference.

A herbal Formulation for all aspects of hair care including reducing hair fall, improvement in hair density, volume, thickness, tensile strength, hair shine and scalp conditions such as dandruff, folliculitis and Psoriasis comprising essentially of a therapeutically effective amount of Ipomoea batatas, Trichosanthes Anguina L, Eclipta prostrata, Murraya koenigii, Phyllanthus emblica, Prunus dulcis, Juglans regia, Arachis hypogaea, Linum usitatissimum, Salvia hispanica, Cicer arietinum, Cinnamomum Zeylanicum Blume, Psidium guajava, Trigonella foenum-graecum, Moringa oleifera, Cuminum cyminum, Spinacia oleracea, Citrus X Limon. Thus the formulation has the potential of addressing most scalp and hair related diseases.

The aforesaid herbal formulation broadly comprises of the herbal constituents in the range of Ipomoea batatas 0%-3.5%, Trichosanthes Anguina L 0%-5.3%, Eclipta prostrata 10%-38%, Murraya koenigii 7%-35%, Phyllanthus emblica 5.3%-15.5%, Prunus dulcis 2.5%-12.5%, Juglans regia 0.5%-7.8%, Arachis hypogaea 3.0%-11.5%, Linum usitatissimum 0.75%-7.5%, Salvia hispanica 0.5%-7.0%, Cicer arietinum 0.5%-7.0%, Cinnamomum Zeylanicum Blume 0.5%-7.0%, Psidium guajava 7%-28%, Trigonella foenum-graecum 0.5%-7.0%, Moringa oleifera 5%-15%, Cuminum cyminum 0%-3.0%, Spinacia oleracea 0%-2.5%, Citrus X Limon 0%-2.5%.

The herbal formulation of the present invention could be manufactured using parts of the plants used for preparing the extracts are Ipomoea batatas tuberous roots, Trichosanthes Anguina L cucurbit vegetable , Eclipta prostrata leaves, stem & flowers, Murraya koenigii leaves, Phyllanthus emblica fruit, Prunus dulcis seeds, Juglans regia seeds, Arachis hypogaea seeds, Linum usitatissimum seeds, Salvia hispanica seeds, Cicer arietinum seeds, Cinnamomum Zeylanicum Blume bark, Psidium guajava leaves, Trigonella foenum-graecum seeds, Moringa oleifera leaves, Cuminum cyminum seeds, Spinacia oleracea leaves, Citrus X Limon Fruit.

As another embodiment the herbal formulation of the present invention comprises of the herbal constituents present in the range of Ipomoea batatas 0.5%, Trichosanthes Anguina L 0.5%, Eclipta prostrata 23.5%, Murraya koenigii 20.0%, Phyllanthus emblica 10.0%, Prunus dulcis 6.0%, Juglans regia 3.0%, Arachis hypogaea 6.1%, Linum usitatissimum 3.0%, Salvia hispanica 3.0%, Cicer arietinum 3.0%, Cinnamomum Zeylanicum Blume 3.0%, Psidium guajava 15.2%, Cuminum cyminum 1.2%, Spinacia oleracea 1.0%, Citrus X Limon 1.0%.

A second embodiment the herbal formulation of the present invention comprises of the herbal constituents present in the range of Ipomoea batatas 1.0%, Trichosanthes Anguina L 1.5%, Eclipta prostrata 20.0%, Murraya koenigii 16.5%, Phyllanthus emblica 8.5%, Prunus dulcis 6.0%, Juglans regia 3.0%, Arachis hypogaea 5.8%, Linum usitatissimum 3.0%, Salvia hispanica 3.0%, Cicer arietinum 3.0%, Cinnamomum Zeylanicum Blume 3.0%, Psidium guajava 11.4%, Trigonella foenum-graecum 3.0%, Moringa oleifera 9.1%, Cuminum cyminum 1.2%, Spinacia oleracea 0.5%, Citrus X Limon 0.5%

### BRIEF DESCRIPTION OF ACCOMPANYING DRAWINGS

FIG. 1: Female Pattern Hair Loss—Ludwig Scale Representation

FIG. 2: Male Pattern Hair Loss—Norwood Scale Representation

FIG. 3: Hair Pull Test—Photographic Representation

FIG. 4: Female Subject Hair Density photos from Phototrichogram

FIG. 5: Male Subject Hair Density photos from Phototrichogram

FIG. 6: Hair Density Test data plot for all subjects at the start and week 24 of clinical trial

FIG. 7: Hair Growth Rate data plot for all subjects at the start and week 24 of clinical trial

FIG. 8: Hair Thickness data plot for all subjects at the start and week 24 of clinical trial

FIG. 9: Hair Tensile Strength data plot for all subjects at the start and week 24 of clinical trial

FIG. 10: Hair Fall Data—Comb Test with Bulb data plot for all subjects at the start and week 24 of clinical trial

FIG. 11: Hair Fall Data—Comb Test without Bulb data plot for all subjects at the start and week 24 of clinical trial

FIG. 12: Hair Texture Rating data plot for all subjects at the start and week 24 of clinical trial

FIG. 13: Hair Shine Rating data plot for all subjects at the start and week 24 of clinical trial

FIG. 14: Hair Thinning (10-point Photonumerical Rating) data plot for all subjects at the start and week 24 of clinical trial

FIG. 15: Representative Photographs of hair thinning differences of two male subjects at the beginging and at week 24 of the clinical trial

FIG. 16: Representative Photographs of hair thinning differences of two female subjects at the beginging and at week 24 of the clinical trial

### DETAILED DESCRIPTION

The following presents a detailed description of various embodiments of the present subject matter with reference to the accompanying drawings. The embodiments of the present subject matter are described in detail with reference to the accompanying drawings. However, the present subject matter is not limited to these embodiments which are only provided to explain more clearly the present subject matter to a person skilled in the art of the present disclosure.

As used herein, the singular forms “a”, “an” and “the” are intended to include the plural forms as well, unless expressly stated otherwise. It will be further understood that the terms “includes”, “comprises”, “including” and/or “comprising” when used in this specification, specify the presence of stated features, integers, steps, operations, elements, and/or components, but do not preclude the presence or addition of one or more other features, steps, operations, elements, components, and/or groups thereof. As used herein, the term “and/or” includes any and all combinations of one or more of the described ingredients having similar properties.

The subject matter described herein relates to a method of improving functional and organoleptic properties of natural products and nutraceutical composition(s) thereof for hair care management including reducing hair fall, improving hair strength, hair density, hair shine and scalp conditions such as dandruff, folliculitis and Psoriasis.

The herbal formulation for scalp and hair related disorders and diseases, comprising extracts from Ipomoea batatas, Trichosanthes Anguina L, Eclipta prostrata, Murraya koenigii, Phyllanthus emblica, Prunus dulcis, Juglans regia, Arachis hypogaea, Linum usitatissimum, Salvia hispanica, Cicer arietinum, Cinnamomum Zeylanicum Blume, Psidium guajava, Trigonella foenum-graecum, Moringa oleifera, Cuminum cyminum, Spinacia oleracea, Citrus X Limon.

The preferred embodiment of the present invention, the herbal organoleptic formulation used for scalp and hair related disorders and diseases, comprising of Ipomoea batatas 0%-3.5%, Trichosanthes Anguina L 0%-5.3%, Eclipta prostrata 10%-38%, Murraya koenigii 7%-35%, Phyllanthus emblica 5.3%-15.5%, Prunus dulcis 2.5%-12.5%, Juglans regia 0.5%-7.8%, Arachis hypogaea 3.0%-11.5%, Linum usitatissimum 0.75%-7.5%, Salvia hispanica 0.5%-7.0%, Cicer arietinum 0.5%-7.0%, Cinnamomum Zeylanicum Blume 0.5%-7.0%, Psidium guajava 7%-28%, Trigonella foenum-graecum 0.5%-7.0%, Moringa oleifera 5%-15%, Cuminum cyminum 0%-3.0%, Spinacia oleracea 0%-2.5%, Citrus X Limon 0%-2.5%

As the embodiment of the present invention, the herbal formulation comprises plant extracts of parts of the plants preferably are Ipomoea batatas tuberous roots, Trichosanthes Anguina L cucurbit vegetable, Eclipta prostrata leaves, stem & flowers, Murraya koenigii leaves, Phyllanthus emblica fruit, Prunus dulcis seeds, Juglans regia seeds, Arachis hypogaea seeds, Linum usitatissimum seeds, Salvia hispanica seeds, Cicer arietinum seeds, Cinnamomum Zeylanicum Blume bark, Psidium guajava leaves, Trigonella foenum-graecum seeds, Moringa oleifera leaves, Cuminum cyminum seeds, Spinacia oleracea leaves, Citrus X Limon Fruit.

As the more preferred embodiment of the present invention, the formulation preferably comprising the herbal constituents in the ration of Ipomoea batatas 0.5%, Trichosanthes Anguina L 0.5%, Eclipta prostrata 23.5%, Murraya koenigii 20.0%, Phyllanthus emblica 10.0%, Prunus dulcis 6.0%, Juglans regia 3.0%, Arachis hypogaea 6.1%, Linum usitatissimum 3.0%, Salvia hispanica 3.0%, Cicer arietinum 3.0%, Cinnamomum Zeylanicum Blume 3.0%, Psidium guajava 15.2%, Cuminum cyminum 1.2%, Spinacia oleracea 1.0%, Citrus X Limon 1.0%.

As yet another embodiment, the formulation of the present invention, preferably comprises the herbal constituents in the ratio of Ipomoea batatas 1.0%, Trichosanthes Anguina L 1.5%, Eclipta prostrata 20.0%, Murraya koenigii 16.5%, Phyllanthus emblica 8.5%, Prunus dulcis 6.0%, Juglans regia 3.0%, Arachis hypogaea 5.8%, Linum usitatissimum 3.0%, Salvia hispanica 3.0%, Cicer arietinum 3.0%, Cinnamomum Zeylanicum Blume 3.0%, Psidium guajava 11.4%, Trigonella foenum-graecum 3.0%, Moringa oleifera 9.1%, Cuminum cyminum 1.2%, Spinacia oleracea 0.5%, Citrus X Limon 0.5%.

The herbal formulation used for the treatment of cosmetic condition selected from male pattern baldness, female pattern baldness, male pattern hair loss, female pattern hairloss, Hair fall reduction, hair follicle strength, hair texture, hair growth rate and scalp conditions including dandruff, folliculitis and Psoriasis.

The herbal formulation used for the treatment of hair loss resulting from skin condition selected from Eczema, dermatitis, Psoriasiss or other inflammatory condition and/or due to age. More particularly, the present herbal formulation used for the treatment of male pattern and female pattern baldness.

As an embodiment of the present invention, a method of promoting hair growth and reducing hair loss comprising administering to a subject an effective amount of herbal formulation of the present invention for a time sufficient for hair regrowth and reduce hair fall.

The foregoing and further objects, features and advantages of the present subject matter will become apparent from the following description of exemplary embodiments with reference to the accompanying drawings.

It is to be noted, however, that the appended drawings illustrate only typical embodiments of the present subject matter, and are therefore, not to be considered for limiting of its scope, for the subject matter may admit to other equally effective embodiments.

### Multi Herbal System Requirement

Most single herbs and spices to provide a particular functionality exhibit certain predominant mechanism of action. If we take hair care management, there are many factors that contribute towards hair loss. They include physiological factors, hormonal factors, environmental factors such as exposure to pollution and psychosomatic factors such as stress and general consequences of ageing.

A single herb or spice or plant may deliver one or more active ingredients that can impact a particular hair loss pathway. Due to diversity of physiology among humans, there could be one or more pathways that are deficient in the hair loss pathway. By consuming one herb they may or may not effectively address the needs of a particular individual in managing their hair loss. A multi-herbal system can address these short comings by simultaneously addressing various regulation pathways and therefore the probability of a multi-herbal system to address the hair loss management deficiencies increases significantly.

### Consuming Many Herbs

While consuming multiple herbs would greatly help with addressing hair care management needs of many individuals, the challenges of developing and consuming multiple herbs daily poses several difficulties: 1. Identification of effective herbal formulation of multiple herbs; 2. Most of the herbs could only be effective when consumed in huge quantities lifelong which is practically not possible; 3. The organoleptics of most natural materials are not suitable for daily consumption to deliver benefits continuously, 4. Achieving compliance remains a challenge with herbal formulations. The above are the most primary roadblocks towards development and administration of a multi-herbal formulations from the all the other issues known to a person skilled in the art.

### Quantity of Herbs

The quantity of each of the herbs to deliver the required functionality is often large.

Consuming large quantity of herbs and also herbs that have pungent taste like garlic makes it very difficult to comply. This is due to low solubility of active ingredients in water. Most beneficial properties are delivered to a particular organ in a water-soluble form to deliver the desired functionality.

The multi-herbal system developed by the inventors of the present invention, with the active ingredients in a water soluble form, the current invention delivers all the benefits of multiple herbs in a water soluble form that have shown to delivery benefits to a much larger cross section of subjects who may individually require assistance from specific target mechanisms which can differ from individual to individual. The formulation with multiple herbs addresses all aspects of hair fall including Hair Growth Rate, Hair Density, Hair Volume, Hair thickness, Hair Fall and scalp conditions such as dandruff, folliculitis and Psoriasis

### Herbs & Spices Used in Current Invention

Ipomoea batatas or Sweet potatoes are a great source of beta-carotene. The body converts this compound into vitamin A, which is linked to good hair health. A medium sweet potato (about 114 grams) contains enough beta-carotene to provide more than four times the daily vitamin A needs. Research has shown that vitamin A promotes the production of sebum, which helps keep hair healthy. Further, vitamin A could also speed up the rate of hair growth and encourage the growth of thicker hair, all while preventing other hair follicles from regressing

Trichosanthes Anguina L or Snake Gourd is a vegetable which is a good source of Vitamin A, Vitamin B and Vitamin C. It also contains constituents like flavonoids, carotenoids, phenolic acids. For people suffering from alopecia, snake gourd is said to stimulate the growth of new hair and protect weakening follicles from hair loss. This can be attributed to it is rich mineral and vitamin content, particularly high levels of carotenes, which specifically delivers skin and hair benefits. It is also claimed that snake gourd can reduce the frequency and intensity of dandruff.

Eclipta prostrata or Flase Daisy is known improves blood circulation and is capable of revitalizing the hair follicles and facilitating hair growth. It is also known to show benefits with respect to reduction in dandruff and graying. False daisy plant oils are said to alleviate inflammation when massaged into the scalp or skin. It can also increase the thickness and luster of hair as well as prevent split ends.

Murraya koenigii or Curry leaves are rich in antioxidants. These antioxidants neutralize the free radicals and keep your hair healthy and strong. They are a good source of proteins and beta-carotene. Both of these nutrients prevent hair loss and thinning. The leaves are also rich in amino acids that help strengthen follicles and keep them healthy. Further, hair follicles get clogged due to factors, such as an oily scalp, pollution, and the build-up of products. The clogged follicles leave the scalp feeling irritated, causing itching, dandruff, and hair fall. Curry leaves can effectively treat these problems.

Phyllanthus emblica or Indian Gooseberry, also called Amla fruits have been long used as a hair growth nourishment in traditional Tibetan and Ayurvedic medicine. It has been shown to stimulate proliferation of Dermal Papilla (DP) cells in a concentration-dependent manner, promoting hair growth by effectively enlarging size and prolonging the anagen phase through the proliferative effect on DP cells.

Prunus dulcis or Almonds nourishes and moisturizes the scalp to reduce flakiness, nourishes hair strands, soften them & prevent the damage caused by hair styling products. Almonds contain vitamins B (particularly riboflavin, thiamine and niacin) and E, protein, fibre, calcium, magnesium, iron, phosphorus, zinc and folate, all of which are beneficial to hair health.

Juglans regia or Walnuts contains potassium, omega-3, omega-6, and omega-9 fatty acids which make the hair strong from the root to the tip. Walnuts are very effective to remove dandruff permanently from the scalp. Essential oils present in walnuts are beneficial to improve blood circulation in the scalp and enhance hair strength. Walnuts also offer anti-bacterial properties to address scalp infections.

Arachis hypogaea or ground nut are a rich source of biotin, also known as vitamin B7. They are also a good source of Vitamin E. Biotin and Vitamin E has been shown to not only stimulate hair growth, but also prevent hair loss.

Linum usitatissimum or Flaxseeds are rich in Vitamin E and Omega-3 fatty acids. Rich in Vitamin E, it promotes the health of the scalp by reducing free radical damage. It helps improve circulation in the head, thus promoting hair growth and slowing down hair breakage. It may also help prevent premature greying of hair. Some studies have shown that omega 3 may help prevent hair loss and thinning of hair, as well as dry scalp and even dandruff.

Salvia hispanica or Chia Seeds have a high content of phosphorous, an ingredient thought to encourage hair growth. Chia seeds contain building block of protein, 1-lysine is known to promote hair growth. Chia seeds also contains Omega-3 fatty acids that are critical to help prevent hair loss.

Cicer arietinum or Chick Peas or Besan is rich in antioxidants, folic acid, iron, copper, zinc, magnesium, selenium, and niacin—all of these nourish hair follicles and improve growth. Chick peas are rich in antioxidants which protect the hair from damaging effects of free radicals. They are also rich in proteins which helps to nourish the hair and combat hair loss. Chick peas also contains molybdenum which is known to support the scalp tissue and surrounding hair follicles so the hair has a strong foundation to grow from.

Cinnamomum Zeylanicum Blume or Cinnamon, when applied to the scalp, increases circulation to the hair follicles and encourages the growth of new strands. Cinnamon is also a potent antimicrobial and can prevent buildup on the scalp from common problems that cause hair loss like dandruff and fungal infections.

Psidium guajava or Guava leaves contain vitamin B and C, which help boost collagen activity required for hair growth. The vitamin C content improves collagen activity aiding in hair growth and the lycopene content gives protection from sun's UV rays.

Trigonella foenum-graecum or Fenugreek seeds contains high protein and nicotinic acid content, which are known to be beneficial against hair fall and dandruff, and in treating a variety of scalp issues like dryness of hair, baldness and hair thinning.

Moringa oleifera or drumstick leaves, due to the presence of a multitude of vitamins and minerals, the leaf of the moringa provides hair with protection from damage, may reduce shedding, dandruff, and promote new hair growth.

Cuminum cyminu or Cumin seeds contains some nutrients and vitamins which enable healthy hair growth and also improves the overall health of hair. Cumin helps in reducing hair fall and baldness and also known to improve texture and increase shininess. Cumin seeds encourage hair growth and strengthen hair follicles. Cumin oil also acts as a natural moisturiser and reduces the dryness on the scalp and hence prevents hair fall

Spinacia oleracea or Spinach is rich in vitamins K, A, C, B2, B6, B1, E, manganese, zinc, iron, and omega-3 fatty acids. These nutrients help nourish scalp and hair, ensuring healthy hair growth. The high content of antioxidants in spinach helps avoid hair damage and boosts scalp health. Vitamin B and C contained in spinach are excellent for encouraging healthy hair growth. They speed up the rate of hair growth by increasing collagen and keratin levels. The rich iron content helps increase the supply of oxygen to the hair follicles, ensuring that they stay in top health. Spinach has anti-inflammatory properties that help soothe an aggravated scalp.

Citrus X Limon or Lemon Lemons are rich in vitamin C. This vitamin can improve the rate at which your hair grows by boosting the production of collagen. It helps clean the scalp and unclog hair follicles. Clogging of the follicles often leads to issues, such as dandruff and hair fall. Lemon has antifungal properties which can can help maintain scalp health and curb issues like dandruff and scalp acne.

### Storage of Herbs & Spices, Grinding and Processes for Enhancing Organoleptic Properties of Herbs and Spices

The herbs and spices used in the present invention are being preferably stored under controlled conditions of temperature & humidity and are processed as discussed in detail in the Indian Patent application no: 202041041780. The formulation and the composition of the multiherb extract for current application in hair and/or scalp related illnesses and for a routine hair care regimen i.e. in a hair care management is accordingly modified.

As yet another embodiment of the present invention the instant formulation is based on supplying nutrients to hair follicles and the skin layers surrounding hair follicles. Skin conditions such as psoriasis, Acne, dandruff, eczema and psoriasis may also cause loss of hair.

Acne is a skin condition that occurs when your hair follicles become plugged with oil and dead skin cells. It causes whiteheads, blackheads or pimples. Acne is most common among teenagers, though it affects people of all ages. Effective acne treatments are available, but acne can be persistent.

Eczema, or dermatitis, is a skin condition that causes patches of itchiness, inflammation, swelling, and cracked skin.

Dandruff is a mild form of seborrheic dermatitis. The exact cause of seborrheic dermatitis is not known, but it's likely a combination of things like too much skin oil (sebum) in the oil glands and hair follicles, a type of yeast found on the skin called Malassezia, environmental & lifestyle factors such as stress, cold and dry winter weather, and some hair care products may make dandruff worse.

Psoriasis is an inflammatory condition and therefore, inflammation-fighting foods are recommended. Antioxidants like vitamin C, vitamin E, beta-carotene, and selenium are known to provide relief.

The current formulation can provide a boost and improve hair re-growth as they contain various compounds that reduce inflammation, provide rich sources of anti-oxidants and also other nutrients and minerals that are supplied to the hair follicles and adjoining skin cells to speed up the recuperation processes.

### EXAMPLES

The present invention is represented below by the help of representative examples. The examples do not limit the scope of the present invention and are incorporated for the purposes of disclosing the best mode of the invention and workability.

### Mixed Herbs Formulation

Herbs and spices were purchased and stored as per storage conditions specified in the Indian Patent application no: 202041041780. The following formulation 1 and 2 are summarized in Table 1 and Table 2 and covers the formulations used in the Examples 1 & 2 to demonstrate the various benefits in hair care herbal waters use.

TABLE 1

| | Weight % |
|---|---|
| Herbal Ingredient Scientific Name | Formulation 1 |
| Ipomoea batatas | 0.5% |
| Trichosanthes Anguina L | 0.5% |
| Eclipta prostrata | 23.5% |
| Murraya koenigii | 20.0% |
| Phyllanthus emblica | 10.0% |
| Prunus dulcis | 6.0% |
| Juglans regia | 3.0% |
| Arachis hypogaea | 6.1% |
| Linum usitatissimum | 3.0% |
| Salvia hispanica | 3.0% |
| Cicer arietinum | 3.0% |
| Cinnamomum Zeylanicum Blume | 3.0% |
| Psidium guajava | 15.2% |
| Cuminum cyminum | 1.2% |
| Spinacia oleracea | 1.0% |
| Citrus X Limon | 1.0% |
| Total | 100.0% |

TABLE 2

| | Weight % |
|---|---|
| Herbal Ingredient Scientific Name | Formulation 2 |
| Ipomoea batatas | 1.0% |
| Trichosanthes Anguina L | 1.5% |
| Eclipta prostrata | 20.0% |
| Murraya koenigii | 16.5% |
| Phyllanthus emblica | 8.5% |
| Prunus dulcis | 6.0% |
| Juglans regia | 3.0% |
| Arachis hypogaea | 5.8% |
| Linum usitatissimum | 3.0% |
| Salvia hispanica | 3.0% |
| Cicer arietinum | 3.0% |
| Cinnamomum Zeylanicum Blume | 3.0% |
| Psidium guajava | 11.4% |
| Trigonella foenum-graecum | 3.0% |
| Moringa oleifera | 9.1% |
| Cuminum cyminum | 1.2% |
| Spinacia oleracea | 0.5% |
| Citrus X Limon | 0.5% |
| Total | 100.0% |

### Example 1

Mixed herbal powder Formulation 1 summarized in Table 2 was used in the test. 14 kg of this mixed herbal powder was dispersed in 56 litres of water. The slurry was kept in suspension with a motorized impeller for 12 hours. The resultant slurry was charged to a 100 litre glass batch distillation still along condenser with a 2.5 sq m condensing surface area. The condenser was water cooled where water circulating at 3 deg C inlet and 5 deg c outlet temperature. The glass vessel was immersed in a 50/50 ethylene glycol batch and heat input to the ethylene glycol was provided by an electrical heating element which was set at 150 deg C. The process was operated for 5 hours at a vacuum of 0.1 mm Hg (0.1 torr) and 30 litres of the herbal concentrate was collected. The boiling point of the batch was 41 deg C. Overall process yield from herbal water to herbal condensate was 53.6%. The herbal condensate was crystal clear and free of any color.

Herbal extract produced was used as an adjunctive supplement in hair care as the ingredients used in the formulation are well known to be effective in various aspects of hair care.

An open label, monocentric, safety and efficacy study to evaluate the safety and efficacy of hair health including hair growth, density, follicle strength and hair fall benefits in healthy subjects was undertaken. Total of 67 subjects were screened and 40 subjects were enrolled in the study and 36 subjects completed the study. Among 36 subjects 25 were female subjects and 11 were male subjects.

Subject selection inclusion and exclusion criteria was as follows:

### Inclusion Criteria

Male and female adult subjects in general good health as determined from a recent medical history, general physical examination, dermatological assessment.
Subjects in the age group of 35-50 years (both ages inclusive).
Subjects complaining of hair fall and damage.
Hair density measurement between 150-250 hair follicle per square cm as per Trichoscan measurement.
Male & female subjects falling under Grade 3-Grade 6 of hair loss severity grade evaluated as per photo numerical 10-point scale (Linear scale for assessment purpose-In use Scale).
Subjects with normal thyroid range.
Subjects who agrees not to use any other product/treatment/home remedy for hair except the provided product during the study period.
Subjects willing to refrain from any type of hair treatment like perming, straightening etc. during the study duration.
Subjects who agree to refrain from using hair dye/hair colour during the study period.
Subjects who are willing to refrain from any other oil treatment/hair spa and oral medications for hair growth during the study.
Subjects willing to give a voluntary written informed consent, photography release and agree to come for regular follow-up.
Subjects willing to abide by and comply with the study protocol.

### Exclusion Criteria

Subjects who have undergone hair growth treatment within 3 months before screening into the study.
Subjects having any active scalp disease which may interfere in the study—dermatologist's judgement.
Subjects who are anaemic (based on RBC count).
Subjects who smoke or drink.
Subjects who are on crash dieting.
Subjects on oral medications such as vitamin supplements, including multi-vitamins which will compromise the study.
Subjects under medical treatment for hair problems, prescription drugs such as Minoxidil, Finasteride, or any 5 α-reductase inhibitor or light therapy.
Subjects who have taken chemotherapy for cancer in the 6 months prior to start of the study or have a plan to do treatments during study.
Subjects who have history of alcoholism, smoking, crash dieting and/or psychiatric disorder including trichotillomania.
Subjects who have had hair transplant, who have taken pharmaceutical product which cause hirsutism (ex. phenytoin) and finesteride for androgenic alopecia, under medical treatment for hair problems.
A known history or present condition of allergic response/hypersensitivity to any cosmetic ingredients and pharmaceutical products.
Subjects on oral medications, undergoing any chemical hair salon treatment-straightening/perming/colour which will compromise the study.
Subjects who are pregnant or lactating or nursing as established with medical history.
Menopausal female subjects as determined by medical history.
Subjects with chronic illness which may influence the cutaneous state.
Subjects participating in other similar cosmetic or therapeutic trial within last three months.
Subjects with any history of underlying uncontrolled medical illness including diabetes mellitus, hypertension, HIV, hepatitis, severe anemia, serious disorder of heart and respiratory apparatus or any other serious medical illness.

### Herbal Water Administration

Each subject was administered 15 ml of herbal water described in Example 1 which was mixed with 500 ml of water and consumed throughout the day for 180 days.

### Subject Investigation Schedule

Visit 1: Pre-screening: The subjects will be required to visit the site for blood collection for CBC and thyroid condition.

Visit 2: Screening visit, shaving and baseline assessment visit (day 1)

Visit 3: Baseline assessment—Hair Density measurement (day 3) (to qualify a subject on inclusion criteria point 4.)

Visit 4: Shaving and assessment visit (day 60)

Visit 5: Assessment visit (day 62)

Visit 6: Shaving and assessment visit (day 120)

Visit 7: Assessment visit (day 122)

Visit 8: Shaving and assessment visit (day 178)

Visit 9: Assessment visit (day 180)

### Parameters Investigated

Efficacy assessment parameters monitored during the study were as follows:
Hair growth rate and hair density using Trichoscan
Hair fibre thickness using Dinolite.
Hair fall assessment by hair comb test, pull test.
Hair strength assessment by tensile test.
Hair fibre thinning assessment by dermatological assessment using global images.
Dermatological assessment on hair quality and site application reaction.
Health and fitness Monitoring

The study was registered CTRI (Clinical Trial Registry of India) after independent ethics committee approval of the study and before enrolment of first subject.

### Tests Methods

### Comb Test

When the hair is detangled with comb as a usual practice, it results in hair fall due to combing force.

The fallen hair can be characterized in two categories:
1. From the root—the hair fibre seen with bulb. These are generally fibres in the telogen phase of the
hair cycle that fall off easily.
2. From the shaft—the breakage of hair from shaft is a direct result of applied force on weaker and damaged hair. This can be identified by a shorter hair (shorter than actual hair length) without any bulb.

This test was performed on visits 2, 4 ,6 and 8.

### Methodology of Hair Combing:

Subjects are asked to partition the hair in two equal halves left and right. They are given a standard tooth comb, to comb each half of the hair in continuous stroking. Number of strokes for each half of
the scalp is fixed (to 10 strokes). The fallen hair is collected on a white sheet and are counted as with and without bulb.
   Data recorded:
Count of total number of hair fall with bulb.
Count of total number of hair fall without bulb.

### Hair Pull Test

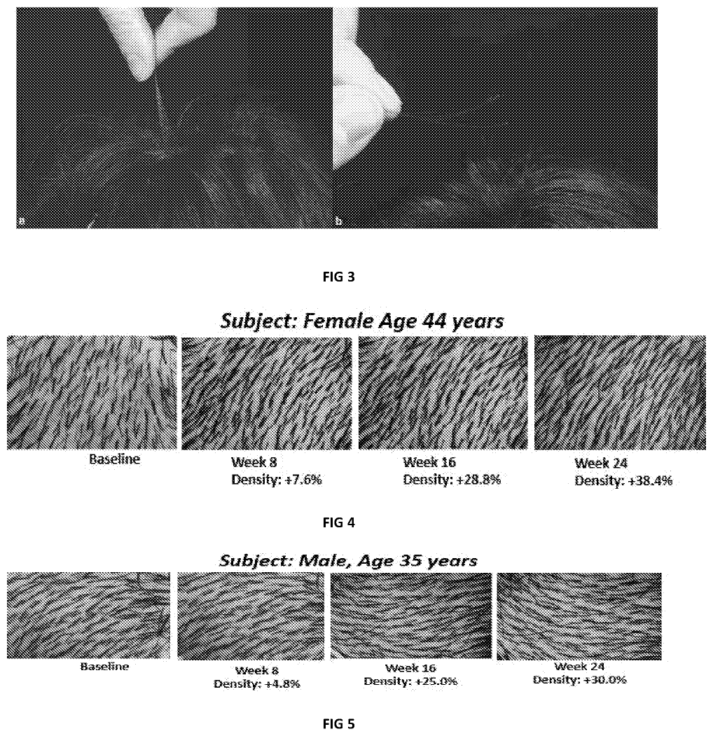

From the vertex of the scalp, a bundle of about 50-60 hair is grasped between the thumb, index finger, and middle finger from the base near the scalp. The hair is firmly, but not forcibly, tugged away from the scalp as fingers slide along the hair shaft. Afterwards the number of extracted hair is counted. FIG. 3 shows is a photographic representation of the hair pull test.

To avoid the biased due to applied force, the assessment was performed by trained evaluators only under dermatologist's supervision. The subjects washed their hair in a manner that there was a minimum of 24 hrs gap between the last wash and the next visit.

This test was performed on visits 2, 4,6 and 8. The scores were given based on the following attributes as summarized in Table 3.

TABLE 3

| Score | Condition | (No. Hair strands) |
|---|---|---|
| 1 | None | (0) |
| 2 | Negligible | (1-2) |
| 3 | Mild | (3-4) |
| 4 | Moderate | (5-6) |
| 5 | Severe | (more than 6) |

### Hair Thinning Measurements

The hair thinning was graded using a 10-point photo numerical linear scale for male & female subjects on all assessment visits by the Dermatologists.

Norwood scale for male subjects (7-point scale) and Ludwig scale for female subjects was used for assessment. This test was performed on visits 2, 4 ,6 and 8.

### Hair Growth Rate Measurements

The assessment was performed by Phototrichogram assessment using Cutiscope on Trichoscan® software. An area on the vertex of the scalp was shaved by trimmer and captured using microscopic camera. This is a quantitative assessment technique to count hair growth rate as p.m/day. The assessment using Phototrichogram was performed on all visits except screening.

### Hair Density Measurements

The assessment was performed by Phototrichogram assessment using Cutiscope on Trichoscan® software. An area on the vertex of the scalp will be shaved by trimmer and captured using microscopic camera. Number of follicles in the area was counted by the software. An increased follicular density could be a result of prolonged hair cycle in anagen phase with less hair fall or higher rejuvenation of follicles. This assessment was performed on visit 3, 5,7 and 9. This is a quantitative assessment technique to count hair density as number of hair/cm2.

### Hair Thickness Measurements

Hair thickness assessment was performed using Dinolite camera using Caselite software. This assessment was performed on visit 2, 4 ,6 and 8.

### Hair Tensile Strength

Three hair samples were used to measure the hair tensile strength of the hair fiber. Three hair fibers or more were cut from each subject to perform tensile test. The hair fiber was attached in root to tip alignment and was pulled at a fixed force and speed. The force at which the hair breaks was recorded and analyzed. Higher the break force, higher the tensile strength of the hair. The method involved using Chatillon Motorized Force Tester. This assessment was performed on visit 2, 4 ,6 and 8.

### Data analysis and Statistics

Table 4 below sumamrises the statistical analysis of the data from the clinical trial

TABLE 4

| | Statistical | | Significance |
|---|---|---|---|
| Statistics | test used | Description | level |
| Normality | Shapiro-Wilk | p-value from Shapiro-Wilk | 5% |
| Checking | Test | normality test at baseline | |
| Efficacy | Paired t-test/ | Mean Value comparison | 5% |
| Checking | Wilcoxon signed | | |
| | rank test for | | |
| | paired samples | | |

All statistical tests used significance level of α≤0.5. Two tailed test was performed for all analysis. All p-values were rounded to 4 decimal places. All p-values that round to 0.000 was presented as ‘<0.0001’ and p-values that round to 1.000 was be presented as ‘>0.9999’. Any p-value ≤0.05 was considered statistically significant as sumamrised below:
Suggestively significant for p-values 0.05<p<0.09
Statistically significant for p-value ≤0.05

### Data Summary

### Hair Density

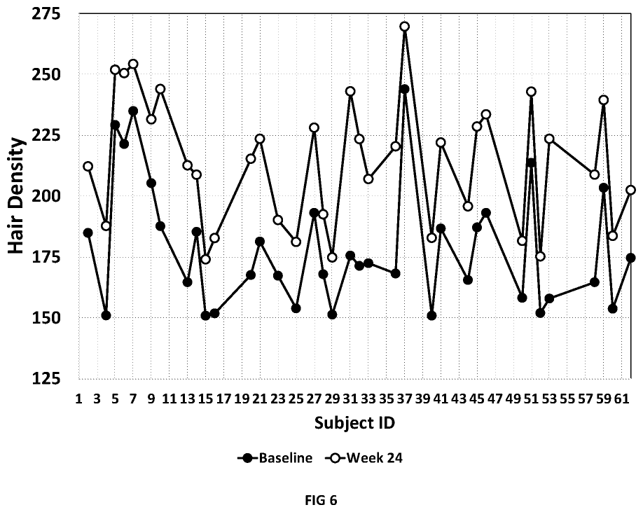

The assessment was performed by Phototrichogram assessment using Cutiscope on Trichoscan® software. An area on the vertex of the scalp was shaved by trimmer and captured using microscopic camera. Number of follicles in the area was counted by the software for hair density. An increased follicular density could be a result of prolonged hair cycle in anagen phase with less hair fall or higher rejuvenation of follicles. Increase in mean value is sign of improvement in hair density. Table 5 sumamrises the hair density test values. FIGS. 4 and 5 shows photographs of two clinical trial participants' hair density photos from phototrichogram along with measured density values. FIG. 6 is plot of hair density values of each subject at the commencement of the trial and at the end of the week 24 of the trial.

TABLE 5

| | Baseline | Week 8 | Week 16 | Week 24 |
|---|---|---|---|---|
| All Subjects | | | | |
| Average | 178.98 | 188.70 | 201.34 | 213.90 |
| Change vs. baseline | | 5.4% | 12.5% | 19.5% |
| p-value | | <0.0001 | <0.0001 | <0.0001 |
| Female Subjects | | | | |
| Average | 178.05 | 188.27 | 200.53 | 212.71 |
| Change vs. baseline | | 5.7% | 12.6% | 19.5% |
| p-value | | <0.0001 | <0.0001 | <0.0001 |
| Male Subjects | | | | |
| Average | 181.08 | 189.67 | 203.17 | 216.60 |
| Change vs. baseline | | 4.7% | 12.2% | 19.6% |
| p-value | | <0.0001 | <0.0001 | <0.0001 |

Data on hair density clearly demonstrates that with daily intake of herbal water, significant improvement was noted in hair density at all-time points in comparison with baseline. Every subject's hair density at the end of the trial was higher than at the start of the trial, thereby indicating that the herbal water was 100% effective among all 36 clinical trial participants. The herbal water exhibited similar performance among male and female subjects.

### Hair Growth Rate

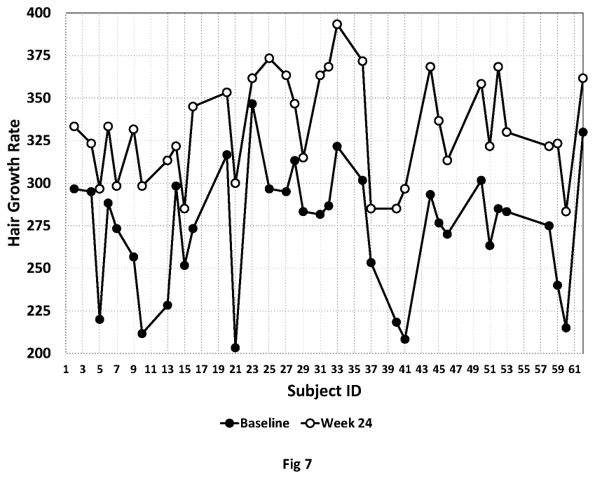

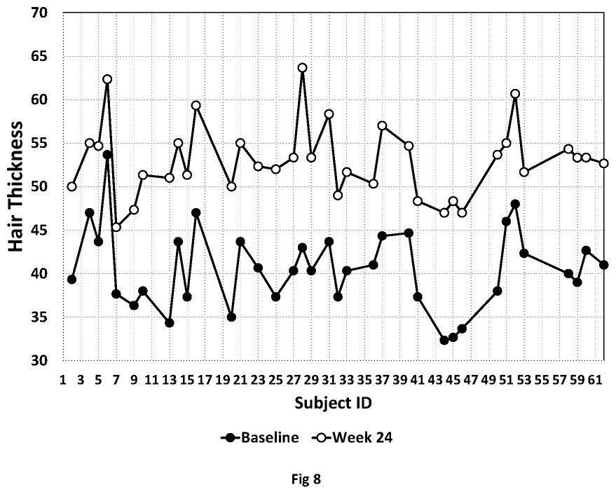

The assessment was performed by Phototrichogram assessment using Cutiscope on Trichoscan® software. An area on the vertex of the scalp was shaved by trimmer and captured using microscopic camera. This was a quantitative assessment technique to count hair growth rate as μm/day. Increase in mean value is sign of improvement in hair growth rate. Table 6 summarises hair growth rate data for the subjects. FIG. 7 is plot of hair growth rate values of each subject at the commencement of the trial and at the end of the week 24 of the trial.

TABLE 6

| | Baseline | Week 8 | Week 16 | Week 24 |
|---|---|---|---|---|
| All Subjects | | | | |
| Average | 273.70 | 287.36 | 311.53 | 331.76 |
| Change vs. baseline | | 5.0% | 13.8% | 21.2% |
| p-values | | <0.001 | <0.001 | <0.001 |
| Female Subjects | | | | |
| Average | 276.00 | 290.80 | 313.20 | 332.60 |
| Change vs. baseline | | 5.4% | 13.5% | 20.5% |
| p-values | | <0.001 | <0.001 | <0.001 |
| Male Subjects | | | | |
| Average | 268.48 | 279.55 | 307.73 | 329.85 |
| Change vs. baseline | | 4.1% | 14.6% | 22.9% |
| p-values | | <0.001 | <0.001 | <0.001 |

Data on hair growth rate clearly demonstrates that with daily intake of herbal water, significant improvement was noted in hair growth rate was measured at all-time points in comparison with baseline. Every subject's hair growth rate at the end of the trial was higher than at the start of the trial, thereby indicating that the herbal water was 100% effective among all 36 clinical trial participants. The herbal water exhibited similar performance among male and female subjects.

### Hair Thickness

Hair thickness assessment was performed using Dinolite camera using Caselite software. Increase in mean value is sign of improvement in hair thickness. Table 7 sumamrises hair thickness data for the subjects. FIG. 8 is plot of hair thickness values of each subject at the commencement of the trial and at the end of the week 24 of the trial.

TABLE 7

| | Baseline | Week 8 | Week 16 | Week 24 |
|---|---|---|---|---|
| All Subjects | | | | |
| Average | 40.54 | 46.31 | 49.59 | 53.02 |
| Change vs. baseline | | 14.2% | 22.3% | 30.8% |
| p-values | | <0.0001 | <0.0001 | <0.0001 |
| Female Subjects | | | | |
| Average | 40.09 | 45.94 | 49.10 | 52.47 |
| Change vs. baseline | | 14.6% | 22.5% | 30.9% |
| p-values | | <0.0001 | <0.0001 | <0.0001 |
| Male Subjects | | | | |
| Average | 41.61 | 47.18 | 50.76 | 54.30 |
| Change vs. baseline | | 13.4% | 22.0% | 30.5% |
| p-values | | <0.0001 | <0.0001 | <0.0001 |

Data on hair thickness clearly demonstrates that with daily intake of herbal water, significant improvement was noted in hair thickness was measured at all-time points in comparison with baseline. Every subject's hair thickness at the end of the trial was higher than at the start of the trial, thereby indicating that the herbal water was 100% effective among all 36 clinical trial participants. The herbal water exhibited similar performance among male and female subjects.

### Hair Tensile Strength

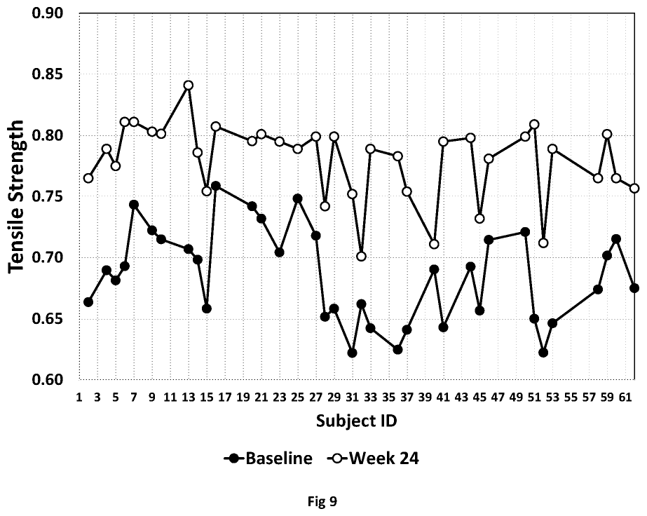

Three hair fibers or more were cut from each subject to perform tensile test. The hair fiber was attached in root to tip alignment and was pulled at a fixed force and speed. The force at which the hair breaks was recorded and analyzed. Higher the break force, higher the tensile strength of the hair. The method involved using Chatillon Motorized Force Tester. Increase in mean value is sign of improvement in hair tensile strength. Table 8 summarises hair tensile strength data for the subjects. FIG. 9 is plot of hair tensile strength values of each subject at the commencement of the trial and at the end of the week 24 of the trial.

TABLE 8

| | Baseline | Week 8 | Week 16 | Week 24 |
|---|---|---|---|---|
| All Subjects | | | | |
| Average | 0.69 | 0.72 | 0.76 | 0.78 |
| Change vs. baseline | | 4.9% | 10.3% | 13.7% |
| p-value | | <0.0001 | <0.0001 | <0.0001 |
| Female Subjects | | | | |
| Average | 0.68 | 0.72 | 0.75 | 0.78 |
| Change vs. baseline | | 4.9% | 10.3% | 13.9% |
| p-value | | <0.0001 | <0.0001 | <0.0001 |
| Male Subjects | | | | |
| Average | 0.69 | 0.72 | 0.76 | 0.78 |
| Change vs. baseline | | 5.0% | 10.4% | 13.3% |
| p-value | | <0.0001 | <0.0001 | <0.0001 |

Data on hair thickness clearly demonstrates that with daily intake of herbal water, significant improvement was noted in hair tensile strength that was measured at all-time points in comparison with baseline. Every subject's hair tensile strength at the end of the trial was higher than at the start of the trial, thereby indicating that the herbal water was 100% effective among all 36 clinical trial participants. The herbal water exhibited similar performance among male and female subjects.

### Comb Test

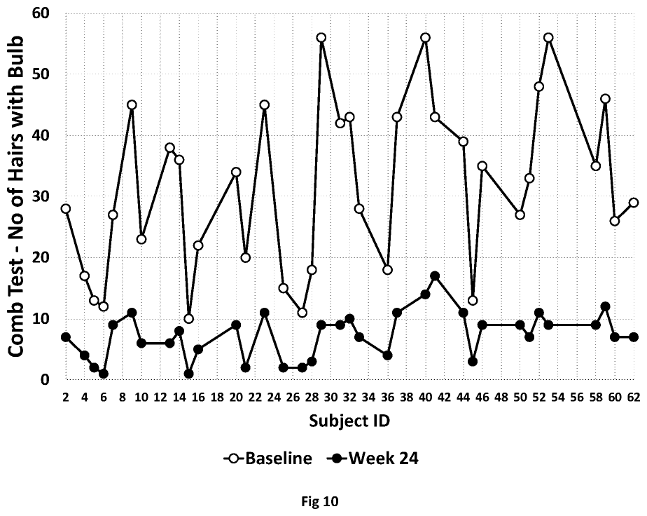

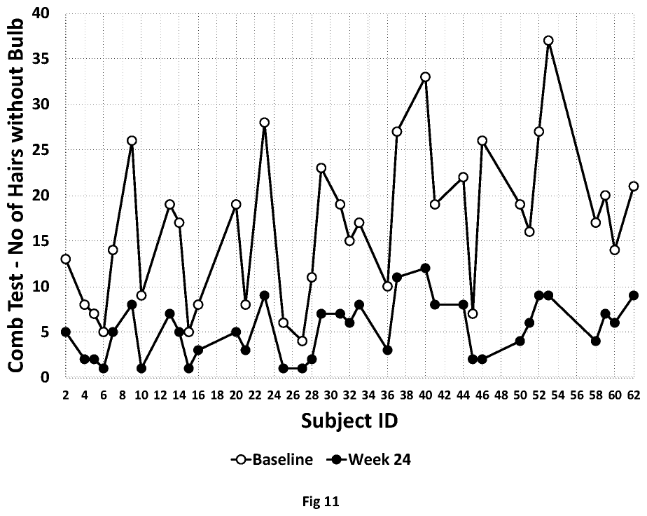

Subjects were asked to partition the hair in two equal halves left and right. A standard tooth comb was provided to comb each half of the hair in continuous stroking. Number of strokes for each half of the scalp was fixed (to may be 10 strokes). The fallen hair was collected on a white sheet and were counted as with bulb and without bulb. Decrease in mean value is sign of improvement in hair fall. Table 9 summarises hair comb test data for the subjects. FIG. 10 is plot of hair comb test values with bulb of each subject at the commencement of the trial and at the end of the week 24 of the trial. FIG. 11 is plot of hair comb test values with without bulb of each subject at the commencement of the trial and at the end of the week 24 of the trial.

TABLE 9

| | Number of hair with bulb | | | | Number of hair without bulb | | | |
|---|---|---|---|---|---|---|---|---|
| | Baseline | Week 8 | Week 16 | Week 24 | Baseline | Week 8 | Week 16 | Week 24 |
| All Subjects | | | | | | | | |
| Average | 31.54 | 20.35 | 10.86 | 7.43 | 16.46 | 12.73 | 7.62 | 5.24 |
| Change vs. baseline | | −35.5% | −65.6% | −76.4% | | −59.6% | −75.8% | −83.4% |
| p-values | | <0.0001 | <0.0001 | <0.0001 | | <0.0001 | <0.0001 | <0.0001 |
| Female Subjects | | | | | | | | |
| Average | 38.35 | 24.69 | 13.46 | 9.42 | 20.35 | 15.42 | 9.54 | 6.73 |
| Change vs. baseline | | −35.6% | −64.9% | −75.4% | | −59.8% | −75.1% | −82.4% |
| p-values | | <0.0001 | <0.0001 | <0.0001 | | <0.0001 | <0.0001 | <0.0001 |
| Male Subjects | | | | | | | | |
| Average | 15.45 | 10.09 | 4.73 | 2.73 | 7.27 | 6.36 | 3.09 | 1.73 |
| Change vs. baseline | | −34.7% | −69.4% | −82.4% | | −58.8% | −80.0% | −88.8% |
| p-values | | <0.0001 | <0.0001 | <0.0001 | | <0.0001 | <0.0001 | <0.0001 |

Data on comb test clearly demonstrates that with daily intake of herbal water, significant improvement was noted in hair fall (with and without bulb) at all time points in comparison with baseline. Every subject's hair fall (with and without bulb) at the end of the trial was higher than at the start of the trial, thereby indicating that the herbal water was 100% effective among all 36 clinical trial participants. The herbal water exhibited similar performance among male and female subjects.

### Hair Pull Test

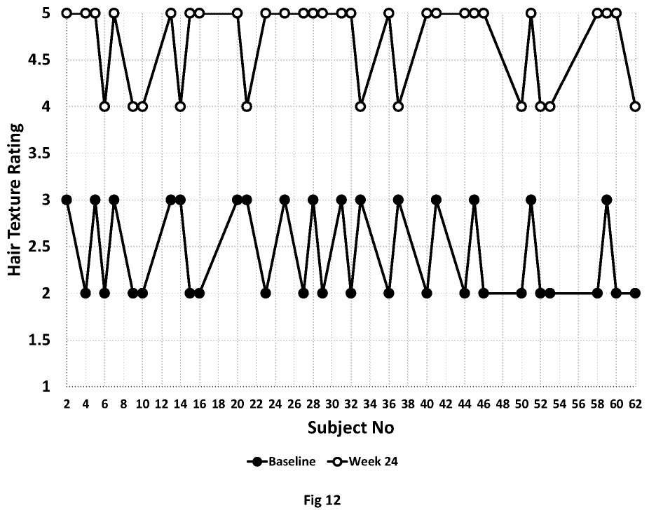

From the vertex of the scalp, a bundle of about 50-60 hair was grasped between the thumb, index finger, and middle finger from the base near the scalp. The hair was firmly, but not forcibly, tugged away from the scalp as fingers slide along the hair shaft. Afterwards the number of extracted hairs was counted. Decrease in mean value is sign of improvement in hair fall. Table 10 summarises hair pull test data for the subjects. FIG. 12 is plot of hair pull test values of each subject at the commencement of the trial and at the end of the week 24 of the trial.

TABLE 10

| | Baseline | Week 8 | Week 16 | Week 24 |
|---|---|---|---|---|
| All Subjects | | | | |
| Average | 2.57 | 2.24 | 1.82 | 1.30 |
| Change vs. baseline | | −12.6% | −28.9% | −49.2% |
| p value | | <0.0001 | <0.0001 | <0.0001 |
| Female Subjects | | | | |
| Average | 2.73 | 2.33 | 1.84 | 1.33 |
| Change vs. baseline | | −14.8% | −32.7% | −51.4% |
| p value | | <0.0001 | <0.0001 | <0.0001 |
| Male Subjects | | | | |
| Average | 2.18 | 2.05 | 1.80 | 1.25 |
| Change vs. baseline | | −6.3% | −17.7% | −42.7% |
| p value | | <0.0001 | <0.0001 | <0.0001 |

Data on hair pull test clearly demonstrates that with daily intake of herbal water, significant improvement was noted in reduction in hair fall at all time points in comparison with baseline. The herbal water exhibited similar performance among male and female subjects.

### Dermatological Assessments

Evaluation was done on comparative scales prepared using hair swatches to aid in subjective assessment. Higher mean value is sign of improvement

### Hair Texture

The following scale was used for rating hair texture changes during the course of the clinical trial: Scale: −1=Very rough hair, 2=Rough hair, 3=Average hair texture (neither rough nor smooth), 4=Smooth hair, 5=Very smooth hair

Table 11 summarises the hair texture ratings among the clinical trial subjects. FIG. 12 shows the hair texture rating plots for all subjects at the start of the clinical trial and at the end of week 24 of the trial.

TABLE 11

| | Baseline | Week 8 | Week 16 | Week 24 |
|---|---|---|---|---|
| All Subjects | | | | |
| Average | 2.43 | 3.00 | 4.19 | 4.70 |
| Change vs. baseline | | 23.3% | 72.2% | 93.3% |
| p-value | | <0.0001 | <0.0001 | <0.0001 |
| Female Subjects | | | | |
| Average | 2.42 | 3.00 | 4.23 | 4.69 |
| Change vs. baseline | | 23.8% | 74.6% | 93.7% |
| p-value | | <0.0001 | <0.0001 | <0.0001 |
| Male Subjects | | | | |
| Average | 2.45 | 3.00 | 4.09 | 4.73 |
| Change vs. baseline | | 22.2% | 66.7% | 92.6% |
| p-value | | <0.0001 | <0.0001 | <0.0001 |

Data on hair texture ratings demonstrate that with daily intake of herbal water, significant improvement was noted in hair texture at all time points in comparison with baseline. Every subject's hair texture at the end of the trial was higher than at the start of the trial, thereby indicating that the herbal water was 100% effective among all 36 clinical trial participants. The herbal water exhibited similar performance among male and female subjects.

### Hair Shine

The following scale was used for rating hair texture changes during the course of the clinical trial: Scale: 1=No shine, 2=Slightly visible shine, 3=Moderately visible shine, 4=Visible shine, 5=Highly visible shine

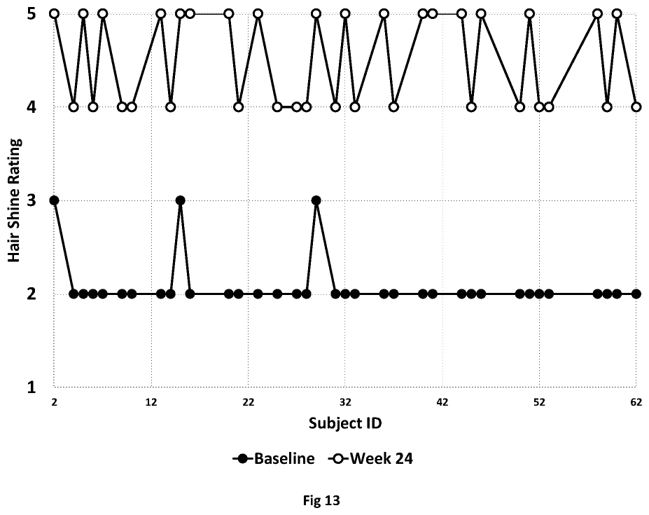

Table 12 summarises the hair shine ratings among the clinical trial subjects during the course of the clinical trial. FIG. 13 shows the hair shine rating plots for all subjects at the start of the clinical trial and at the end of week 24 of the trial.

TABLE 12

| | Baseline | Week 8 | Week 16 | Week 24 |
|---|---|---|---|---|
| All Subjects | | | | |
| Average | 2.08 | 3.00 | 4.14 | 4.51 |
| Change vs. baseline | | 44.2% | 98.7% | 116.9% |
| p-value | | <0.0001 | <0.0001 | <0.0001 |
| Female Subjects | | | | |
| Average | 2.08 | 3.00 | 4.15 | 4.62 |
| Change vs. baseline | | 44.4% | 100.0% | 122.2% |
| p-value | | <0.0001 | <0.0001 | <0.0001 |
| Male Subjects | | | | |
| Average | 2.09 | 3.00 | 4.09 | 4.27 |
| Change vs. baseline | | 43.5% | 95.7% | 104.3% |
| p-value | | <0.0001 | <0.0001 | <0.0001 |

Data on hair shine ratings demonstrate that with daily intake of herbal water, significant improvement was noted in hair shine at all time points in comparison with baseline. Every subject's hair shine at the end of the trial was higher than at the start of the trial, thereby indicating that the herbal water was 100% effective among all 36 clinical trial participants. The herbal water exhibited similar performance among male and female subjects.

### Hair Thinning Based on 10 Point Photo Numerical Linear Scale

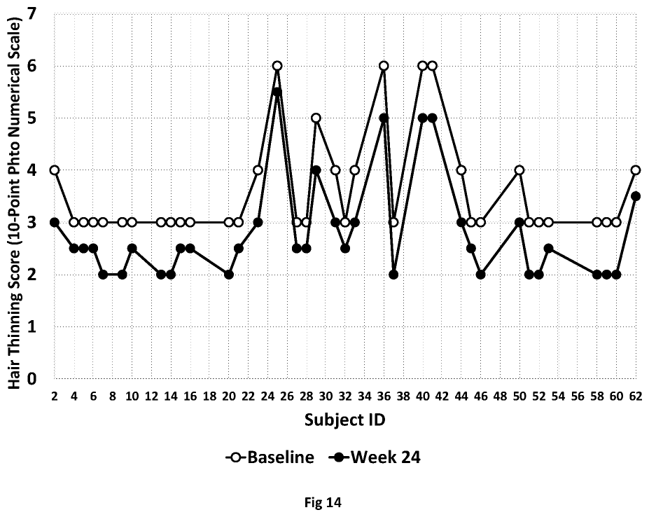

The hair thinning was graded using a 10-point photo numerical linear scale for male & female subjects on all assessment visits by the Dermatologists. Lower value indicates improvement in hair thinning. Table 13 summarises the hair thinning ratings among the clinical trial subjects during the course of the clinical trial. FIG. 14 shows the hair thinning rating plots for all subjects at the start of the clinical trial and at the end of week 24 of the trial.

TABLE 13

| | Baseline | Week 8 | Week 16 | Week 24 |
|---|---|---|---|---|
| All Subjects | | | | |
| Average | 3.62 | 3.42 | 3.07 | 2.81 |
| Change vs. Baseline | | −5.6% | −15.3% | −22.4% |
| p-Value | | <0.0001 | <0.0001 | <0.0001 |
| Female Subjects | | | | |
| Average | 3.65 | 3.38 | 3.02 | 2.73 |
| Change vs. Baseline | | −7.4% | −17.4% | −25.3% |
| p-Value | | <0.0001 | <0.0001 | <0.0001 |
| Male Subjects | | | | |
| Average | 3.55 | 3.50 | 3.18 | 3.00 |
| Change vs. Baseline | | −1.3% | −10.3% | −15.4% |
| p-Value | | 0.3409 | 0.0004 | <0.0001 |

Data on hair thinning demonstrate that with daily intake of herbal water, significant improvement was noted in hair thinning at all time points in comparison with baseline. Every subject's thinning at the end of the trial improved than at the start of the trial, thereby indicating that the herbal water was 100% effective among all 36 clinical trial participants. The herbal water exhibited better performance among female than male subjects. The hair thinning score among female subjects was statistically significant by week 8 of the trial whereas for male subjects the data achieved suggestively significant between week 8 and week 16 and statistically significant from week 16 onwards.

### Hair Thinning Based on Ludwig Scale (Female Subjects)

Assessments on hair thinning as per Ludwig Scale was performed. FIG. 1 schematically represents the Ludwig Scale. In this assessment, Lower grade indicates improvement in hair thinning. Table 14 summarises the Ludwig Scale hair thinning assessments conducted during the course of the clinical trial.

TABLE 14
Hair Thinning data of Female subjects (Frequency)

| | Baseline | Week 8 | Week 16 | Week 24 |
|---|---|---|---|---|
| I-I | 0 | 1 | 8 | 12 |
| I-2 | 12 | 13 | 6 | 7 |
| I-3 | 6 | 5 | 6 | 2 |
| I-4 | 4 | 3 | 2 | 2 |
| II-1 | 3 | 3 | 3 | 2 |
| II-2 | 1 | 1 | 1 | 1 |

Observation:
1. At Week 8: 1 subject was noted to shift from 1-4 to 1-3, 2 subjects were noted to shift from 1-3 to 1-2 and 1 subject was noted to shift from 1-2 to I-I implying reduction in hair thinning
2. At Week 16: 1 subject was noted to shift from 1-4 to 1-3. 7 subjects were noted to shift from 1-2 to I-I implying reduction in hair thinning
3. At Week 24: 1 subject was noted to shift from II-1 to 1-4. 1 subject was noted to shift from 1-4 to 1-3. 5 subjects were noted to shift from 1-3 to 1-2. 4 subjects were noted to shift from 1-2 to I-I implying reduction in hair thinning

### Hair Thinning Based on Norwood Scale (Male Subjects)

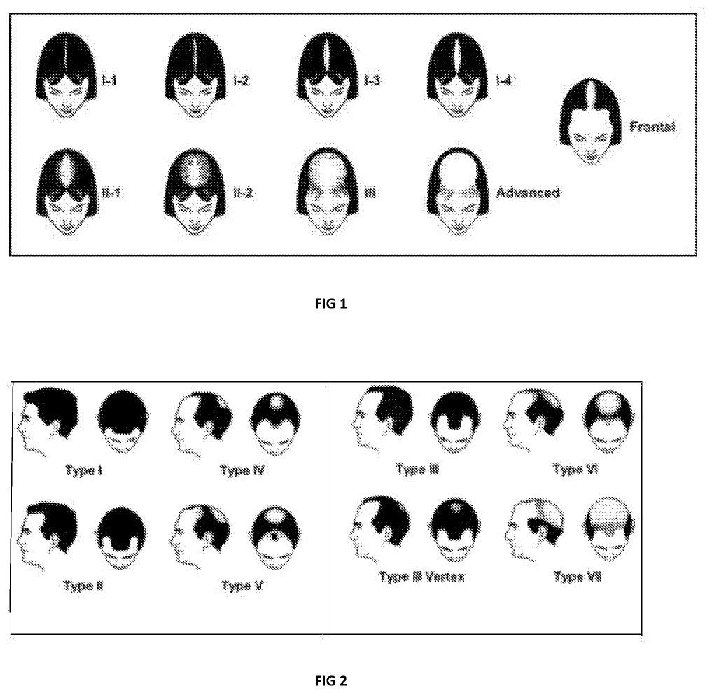

Assessments on hair thinning as per Ludwig Scale was performed. FIG. 2 schematically represents the Ludwig Scale. In this assessment, Lower grade indicates improvement in hair thinning. Table 15 summarises the Ludwig Scale hair thinning assessments conducted during the course of the clinical trial.

TABLE 15

| | Baseline | Week 8 | Week 16 | Week 24 |
|---|---|---|---|---|
| Type I-Grade 1 | 1 | 1 | 1 | 1 |
| Type II = Grade 2 | 1 | 1 | 1 | 1 |
| Type II-Grade 2 | 4 | 4 | 4 | 5 |
| Type III Vertex = Grade 3 | 2 | 2 | 2 | 1 |
| Type III = Grade 3 | 3 | 3 | 3 | 3 |

One (1) subject was noted to shift from Type III vertex=Grade 3 to type II=Grade 2 on the Norwood Scale implying reduction in hair thinning at week 24.

### Hair Thinning Representative Photographs

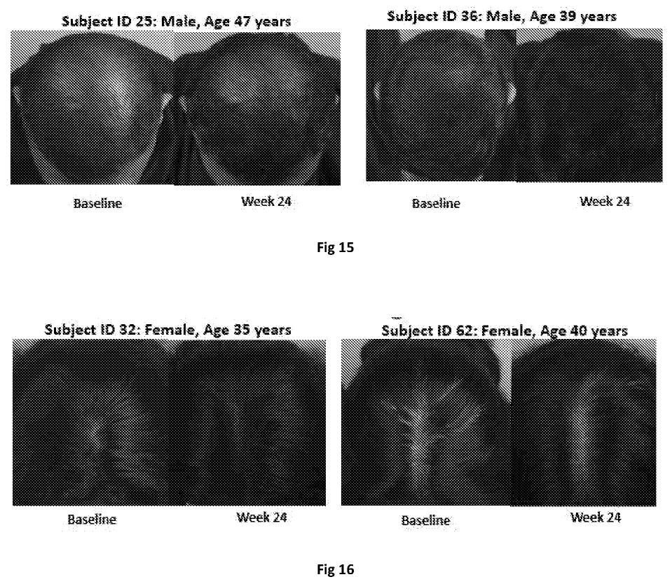

FIGS. 15 & 16 shows representative photographs of Male and Female subjects respectively at the beginning and end of the trial. The data clearly pictorially demonstrates the improvement of hair thinning characteristics at the end of the week 24 of consuming herbal water formulation for hair care.

### Example 2

Mixed herbal powder Formulation 2 summarized in Table 3 was used in the test. 14 kg of this mixed herbal powder was dispersed in 56 litres of water. The slurry was kept in suspension with a motorized impeller for 12 hours. The resultant slurry was charged to a 100 litre glass batch distillation still along condenser with a 2.5 sq m condensing surface area. The condenser was water cooled where water circulating at 3 deg C inlet and 5 deg c outlet temperature. The glass vessel was immersed in a 50/50 ethylene glycol batch and heat input to the ethylene glycol was provided by an electrical heating element which was set at 150 deg C. The process was operated for 5 hours at a vacuum of 0.1 mm Hg (0.1 torr) and 30 litres of the herbal concentrate was collected. The boiling point of the batch was 40.5 deg C. Overall process yield from herbal water to herbal condensate was 53.6%. The herbal condensate was crystal clear and free of any color.

Herbal extract produced was used as an adjunctive supplement in hair care as the ingredients used in the formulation are well known to be effective in various aspects of hair care.

A study with 10 subjects to evaluate the safety and efficacy of hair health using hair fall benefits in healthy subjects was undertaken. Each subject consumed 15 ml of herbal water mixed with 500 ml of water and consumed throughout the day for a period of 16 weeks.

Hair fall was quantified among the ten subjects with the comb test described in detail in Example 1. Hair fall was measured with and without the bulb. Table 16 summarizes data on hair fall during the course of the trial.

TABLE 16

| | Number of hair with bulb | | | Number of hair without bulb | | |
|---|---|---|---|---|---|---|
| | Baseline | Week 8 | Week 16 | Baseline | Week 8 | Week 16 |
| All Subjects | | | | | | |
| Average | 35.00 | 23.00 | 10.32 | 18.30 | 11.76 | 7.32 |
| Change vs. baseline | | −34.3% | −70.5% | | −35.7% | −60.0% |
| p-values | | <0.0001 | <0.0001 | | <0.0001 | <0.0001 |

Data on comb test clearly demonstrates that with daily intake of herbal water, significant improvement was noted in hair fall (with and without bulb) at all time points in comparison with baseline. Every subject's hair fall (with and without bulb) at the end of the trial was higher than at the start of the trial, thereby indicating that the herbal water was 100% effective among all 10 participants.

By combining multiple herbs into an easily consumable water format with good organoleptic properties in addition to good functional properties, it allows easy consumption of the herbal waters on a daily basis by subjects.

The significant improvements on all aspects of hair care including hair density, hair thickness, hair fall, hair tensile strength, hair thinning among 100% of the 36 subjects who completed the test indicates the herbal water formulation delivers required efficacy. The product performance attributes were similar for male and female subjects.

### Example 3

The performance of herbal eater extract of the present invention is compared against Minoxidil and a herbal supplement in a capsule form under the brand name Nutrafol®.

Minoxidil (2,4-diamino-6-piperidinopyrimidine 3-oxide), is a medication used for the treatment of high blood pressure and pattern hair loss in males and females. It is an antihypertensive vasodilator which is also believed to improve blood flow to the scalp. It is available as a generic medication by prescription in oral tablet form and over the counter as a topical liquid or foam.

Nutrafol® is a Nutraceutical Supplement containing Synergen Complex® (Circumin, Ashwagandha, Saw Palmetto and tocotrenols) administered in a capsule form with 4 capsules administered once per day.

Table 17 compares the clinical data with Minoxidil administered in oral form at 5mg dose and Nutrafol® along with comparative data from clinical trial with the present invention. Hair density changes for men shows equivalent performance of the current invention versus Minoxidil consumed in an oral form. Hair thickness of the present invention is shown to be over two times that of Oral Minoxidil.

The present invention clinical data showed almost twice higher density improvements versus Nurafol® Herbal Supplement.

Further, the present herbal water has been tested to show very similar performance in male and female participants in not only hair density and hair diameter, as summarized in Example 2, hair growth rate, hair tensile strength, hair fall, hair texture has all shown very similar improvements.

TABLE 17
Comparative performance of Oral Minoxidil, Nutrafol ® Supplement &
Herbal Water based Oral Nutraceutical Supplement from present invention

| | | Hair Density in 1 sq cm area | | | | Hair Diameter | | | |
|---|---|---|---|---|---|---|---|---|---|
| | Clinical | | | Week 12 | Week 24 | | | Week 12 | Week 24 |
| Treatment | Trial Details | Day 0 | | (Day 84) | (Day 168) | Day 0 | | (Day 84) | (Day 168) |
| Oral Minoxidil (1) | 30 men consuming | 182.5 | | 208.5 | 217.6 | 58.5 | | 64.7 | 67.4 |
| | Oral Minoxidil, | | | | | | | | |
| | 5 mg for 24 weeks | | | | | | | | |
| | Increase vs Day 0 | | | 14.2% | 19.2% | | | 10.6% | 15.2% |
| | | Day 0 | | Day 90 | Day 180 | | | | |
| Nutraceutical Supplement | Randomized Double | 141.7 | | 151.4 | 156.4 | | | | |
| (Nutrafol ® Capsules) | Blind Study of 40 | | | | | | | | |
| containing Synergen | women | | | | | | | | |
| Complex ® (containing | (treatment n = 26, | | | | | | | | |
| Circumin, Ashwagandha, | Placebo n = 14 | | | | | | | | |
| Saw Palmetto and | | | | | | | | | |
| tocotrenols) (2) | | | | | | | | | |
| | Increase vs Day 0 | | | 6.8% | 10.4% | | | | |
| | | Day 0 | Week 8 | Week 16 | Week 24 | Day 0 | Week 8 | Week 16 | Week 24 |
| Present Invention | Open Label, men (n = | 179 | 188.7 | 201.3 | 213.9 | 40.5 | 46.3 | 49.6 | 53.0 |
| (Herbal Water | 11) & women (n = 26) - | | | | | | | | |
| nutraceutical | All Subjects | | | | | | | | |
| oral consumption) | | | | | | | | | |
| | Increase vs Day 0 | | 5.4% | 12.5% | 19.5% | | 14.2% | 22.3% | 30.8% |
| | Male Subjects (n = 11) | 181.1 | 189.7 | 203.2 | 216.6 | 41.6 | 47.2 | 50.8 | 54.3 |
| | Increase vs Day 0 | | 4.7% | 12.2% | 19.6% | | 13.4% | 22.0% | 30.5% |
| | Female Subjects (n = 26) | 178.1 | 188.3 | 200.5 | 212.7 | 40.1 | 45.9 | 49.1 | 52.5 |
| | Increase vs Day 0 | | 5.7% | 12.6% | 19.5% | | 14.6% | 22.5% | 30.9% |

(1) Panchaprateep, R., Lueangarun, S. Efficacy and Safety of Oral Minoxidil 5 mg Once Daily in the Treatment of Male Patients with Androgenetic Alopecia: An Open-Label and Global Photographic Assessment. Dermatol Ther (Heidelb) 10, 1345-1357 (2020). https://doi.org/10.1007/s13555-020-00448-x
(2) Ablon G, Kogan S. A Six-Month, Randomized, Double-Blind, Placebo-Controlled Study Evaluating the Safety and Efficacy of a Nutraceutical Supplement for Promoting Hair Growth in Women With Self-Perceived Thinning Hair. J Drugs Dermatol. 2018 May 1; 17(5): 558-565. PMID: 29742189.

Although the invention has been described with reference to specific embodiments, this description is not meant to be construed in a limiting sense as it will become apparent to persons skilled in the art upon reference to the description of the invention. It is therefore, contemplated that such modifications can be made without departing from the spirit or scope of the present invention as defined.

1. A herbal formulation for scalp and hair related disorders and diseases, comprising extracts from plant material selected from plants from the group consisting of Ipomoea batatas, Trichosanthes Anguina L, Eclipta prostrata, Murraya koenigii, Phyllanthus emblica, Prunus dulcis, Juglans regia, Arachis hypogaea, Linum usitatissimum, Salvia hispanica, Cicer arietinum, Cinnamomum Zeylanicum Blume, Psidium guajava. Trigonella foenum-graecum, Moringa oleifera, Cuminum cyminum, Spinacia oleracea, Citrus X Limon and combinations thereof.

2. The herbal formulation as claimed in claim 1, comprising

| | |
|---|---|
| Ipomoea batatas | 0%-3.5%, |
| Trichosanthes Anguina L | 0%-5.3%, |
| Eclipta prostrata | 10%-38%, |
| Murraya koenigii | 7%-35%, |
| Phyllanthus emblica | 5.3%-15.5%,, |
| Prunus dulcis | 2.5%-12.5%, |
| Juglans regia | 0.5%-7.8%, |
| Arachis hypogaea | 3.0%-11.5%, |
| Linum usitatissimum | 0.75%-7.5%, |
| Salvia hispanica | 0.5%-7.0%, |
| Cicer arietinum | 0.5%-7.0%, |
| Cinnamomum Zeylanicum Blume | 0.5%-7.0%, |
| Psidium guajava | 7%-28%, |
| Trigonella foenum-graecum | 0.5%-7.0%, |
| Moringa oleifera | 5%-15%, |
| Cuminum cyminum | 0%-3.0%, |
| Spinacia oleracea | 0%-2.5%, and |
| Citrus X Limon | 0%-2.5% |

as weight percent of the herbal formulation.

3. The herbal formulation as claimed in claim 1, wherein the plant material comprises Ipomoea batatas tuberous roots, Trichosanthes Anguina L cucurbit vegetable, Eclipta prostrata leaves, stem and flowers, Murraya koenigii leaves, Phyllanthus emblica fruit, Prunus dulcis seeds, Juglans regia seeds, Arachis hypogaea seeds, Linum usitatissimum seeds, Salvia hispanica seeds, Cicer arietinum seeds, Cinnamomum Zeylanicum Blume bark, Psidium guajava leaves, Trigonella foenum-graecum seeds, Moringa oleifera leaves, Cuminum cyminum seeds, Spinacia oleracea leaves, Citrus X Limon fruit.

4. The herbal formulation as claimed in claim 1, comprising:

| | |
|---|---|
| Ipomoea batatas | 0.5%, |
| Trichosanthes Anguina L | 0.5%, |
| Eclipta prostrata | 23.5%, |
| Murraya koenigii | 20%, |
| Phyllanthus emblica | 10.0%, |
| Prunus dulcis | 6.0%, |
| Juglans regia | 3.0%, |
| Arachis hypogaea | 6.1%, |
| Linum usitatissimum | 3.0%, |
| Salvia hispanica | 3.0%, |
| Cicer arietinum | 3.0% |
| Cinnamomum Zeylanicum Blume | 3.0%, |
| Psidium guajava | 15.2%, |
| Cuminum cyminum | 1.2%, |
| Spinacia oleracea | 1.0%, and |
| Citrus X Limon | 1.0% |

as weight percent of the berhal formulation.

5. The herbal formulation as claimed in claim 1, comprising:

| | |
|---|---|
| Ipomoea batatas | 1.0%, |
| Trichosanthes Anguina L | 1.5%, |
| Eclipta prostrata | 20.0%, |
| Murraya koenigii | 16.5%, |
| Phyllanthus emblica | 8.5%, |
| Prunus dulcis | 6.0%, |
| Juglans regia | 3.0%, |
| Arachis hypogaea | 5.8%, |
| Linum usitatissimum | 3.0%, |
| Salvia hispanica | 3.0%, |
| Cicer arietinum | 3.0%, |
| Cinnamomum Zeylanicum Blume | 3.0%, |
| Psidium guajava | 11.4%, |
| Trigonella foenum-graecum | 3.0%, |
| Moringa oleifera | 9.1%, |
| Cuminum cyminum | 1.2%, |
| Spinacia oleracea | 0.5%, and |
| Citrus X Limon | 0.5% |

as weight percent of the herbal formulation.

6. (canceled)

7. (canceled)

8. (canceled)

9. A method of promoting hair growth and reducing hair loss comprising:
administering to a subject an effective amount of herbal formulation as claimed in claim 1 for a time sufficient for hair treatment or a scalp treatment.

10. The method as claimed in claim 9, wherein the hair treatment is selected from the group consisting of male pattern baldness, female pattern baldness, male pattern hair loss, female pattern hair loss, reduction of hair loss, hair follicle strengthening, hair texture improvement, hair growth rate increase, and combinations thereof.

11. The method as claimed in claim 9, wherein the scalp treatment is selected from the group consisting of eczema, dermatitis, psoriasiss, dandruff, folliculitis, an inflammatory condition, and combinations thereof.

12. The method as claimed in claim 9, wherein the hair treatment is male pattern and/or female pattern baldness.
